(19) United States
(12) Patent Application Publication
(10) Pub. No.: US 2022/0145259 A1
(43) Pub. Date: May 12, 2022

(54) Liver Tissue Model Constructs and Methods for Providing the Same

(71) Applicant: Cellink AB, Göteborg (SE)

(72) Inventors: Duong Nguyen, Göteborg (SE); Adel Itedal Namro Redwan, Skepplanda (SE); Hector Martinez, Göteborg (SE); Erik Gatenholm, Göteborg (SE)

(21) Appl. No.: 17/438,031

(22) PCT Filed: Mar. 13, 2020
(86) PCT No.: PCT/EP2020/056878

§ 371 Date: Sep. 10, 2021

(30) Foreign Application Priority Data
Mar. 13, 2019 (SE) 1950316-8

(51) Int. Cl. C12N 5/071; C12N 5/00; A61L 27/38; A61L 27/26; A61L 27/36; A61L 27/48; B33Y 70/00; B33Y 80/00; B33Y 40/10; B33Y 10/00

(52) U.S. Cl. CPC C12N 5/0671; A61L 2300/802; A61L 27/3886; A61L 27/26; A61L 27/3633; A61L 27/48; A61L 27/3839; A61L 27/3834; A61L 27/3804; A61L 27/3641; B33Y 70/00; B33Y 80/00; B33Y 40/10; B33Y 10/00; C12N 2503/04; C12N 2533/52; C12N 2533/54; C12N 2533/74; C12N 2533/78; A61L 2430/28; A61L 2300/414; C12N 5/0062

(57) ABSTRACT

The present invention provides for a liver tissue model construct composed of biomaterials and cells, to be used for scientific research within in the 3D liver tissue modelling field. The applications of said tissue model construct can be specific for pharmaceutical evaluations and/or discoveries, regenerative medicine investigations, tissue engineering developments, and liver physiology and/or pathology.

### TECHNICAL FIELD

The present invention relates to the field of 3D bioprinting.

### BACKGROUND ART

In the field of in vitro models, the gold standard for cell culture is cells grown on two-dimensional (2D) tissue culture plastic. However, cells within the human body are organized and distributed in three-dimensional (3D) space. Therefore, to provide highly relevant information of cell and human physiology, research must be conducted in 3D tissue models. Due to the importance of the liver function in drug metabolism and blood filtration, a 3D liver tissue model is of high interest for applications in drug development, tissue engineering, and regenerative medicine for both physiological and pathological understanding and evaluation. A 3D liver tissue model with highly relevant human physiological mimicry will improve efficiency of therapeutic and biological development. Hence, ongoing research have investigated spheroids and organoids as 3D models.

US2018016548 A1 discloses a system comprising a bioscaffold comprising acellular hepatic extracellular matrix and induced pluripotent stem cell (iPSC)-derived hepatocytes.

WO2018115533 A1 discloses artificial spheroidal microtissue comprising hepatocytes, and

US2014274802 AA discloses engineered, living, three-dimensional liver tissue constructs.

### SUMMARY OF THE INVENTION

While spheroid and organoid cultures demonstrate hepatic cells in a 3D orientation, limitations such as cells crowding within the defined space, minimal capability of migrating, fragility, and cell number restrictions indicate a need for more advanced 3D tissue models. 3D bioprinting technology, using bioinks and bioprinters, is a platform in which the limitations above have been addressed and refined for the development of an equivalent tissue model of the liver. The object of the present invention is to provide a solution of how to obtain a 3D liver tissue without the limitations mentioned above.

The object is attained as disclosed in the appended claims, wherein an all-inclusive solution is provided for generating a 3D liver tissue model with extended working time as compared to conventional 2D sandwich culture. The present solution also offers a more efficient production as compared to spheroid and organoid cultures. The components of the present invention offers bioinks that compliment most hepatic cell types and disease induction factors, and resolve challenges such as 3D environments compatible with multiple hepatic cell types, compatible co-cultures comprising hepatic cells, effective stimulant models of disease conditions, and protocols for generating medium-throughput 3D tissue models. The bioprinting parameters that are important for generating a 3D model have been optimized in order to facilitate the application of the liver tissue model.

The invention relates to a combination of (i) bioinks, (ii) cells, and (iii) reagents that will provide the users with the tools to customize a 3D liver tissue model for their applications. Furthermore, a kit comprising the bioinks and protocols will provide the user with starting parameters such as cell density, bioprinting code, crosslinking procedure, and concentration of stimulating factor to facilitate the generation of tissue models with 3D bioprinting. Once the constructs have been stabilized in culture, the user is able to conduct other experimental variables of interest.

Thus, in a first aspect, the object above is attained by a tissue model construct comprising at least two 3D bioprinted structures, wherein the first structure comprises a Bioink A mixed with a Cell A, and the second structure comprises a Bioink B mixed with a Cell B,
wherein Bioink A and Bioink B, each and independently of each other, comprises materials including but not limited to biopolymers synthetically or naturally derived from plants and/or animals, such as gelatin, alginate, cellulose, and thickening agents, photo initiators, and/or other extracellular matrix proteins,
wherein Cell A is a hepatic cell line of human or animal origin, or primary hepatic cells, or induced Pluripotent Stem cell (iPS)-derived or Embryonic Stem cell (ES)-derived hepatic cells, and
wherein Cell B is a non-parenchymal cell of human or animal origin from cell lines, primary cells, or derived from induced Pluripotent Stem cells (iPSCs) or Embryonic Stem cells (ESCs).

The Bioink A may comprise methacrylated gelatin or collagen with a liver specific Extra-Cellular Matrix (ECM) component, such as laminins, fibronectin or whole organ digest ECM from animal or human origin.

The Bioink B may comprise liver specific components such as laminins, fibronectin or whole organ digest ECM from animal or human origin.

The thickening agent may be synthetic or natural. Preferably, the thickening agent is a natural polysaccharide chosen from a group comprising xanthan gum, glucomannan and nanocellulose.

Cell A and/or Cell B, each and independently of each other, may be immature or mature non-parenchymal hepatic cells originating from induced pluripotent stem cells, embryonic stem cells, any other stem cells, primary cells, and cell lines of human or animal origin.

The first and/or second structure of the tissue model construct according to the above may be stimulated post printing with the Factor A to stimulate abnormal functions and/or phenotypical changes of Cell A and/or Cell B, wherein Factor A is chosen from the group comprising TGF-β, free fatty acids, lipopolysaccharide in combination with allyl alcohol, and cytokines such as interleukins and tumor necrosis factors. Abnormal functions and/or phenotypical changes may for example be addition of TGF-β to induce fibrosis which can be seen by deposition of collagen, or addition of free fatty acid to generate a fatty liver disease model which can be seen by deposition of fats in the cell by oil red staining.

Bioink A and Bioink B may, each and independently of each other, comprise:
a plant based material 0-90%
an animal/human derived material 0-90%
thickening agents 0-50%
a photo initiator 0-10%
a growth factor specific to parenchymal cells 0-20%.

Cell A and Cell B may be used at different ratios in relation to each other, in order to simulate a pathology in vitro.

The tissue model construct according to the above may be for use within science, medicine, tissue engineering, pharmaceutical therapies, regenerative medicine, stem cell research, and in vitro models.

Furthermore the present disclosure provides for a method of bioprinting the tissue model construct according to the above, wherein the 3D bioprinting is performed with an extrusion based bioprinting device.

Furthermore, the present disclosure provides for use of the tissue model construct according to the above for:
i) developmental biology in order to gain understanding of cellular activities within a 3D environment such as cellular distribution, migration, proliferation, matrix production, interactions with other cells and the surrounding environment, etc.;
ii) pharmaceutical applications for drug discovery, target validation, toxicity studies, metabolic studies, cellular differentiation/maturation, spheroid differentiation/maturation, organoid differentiation/maturation, etc.;
iii) tissue regeneration applications such as tissue remodeling, cellular proliferation, cellular metabolism, cellular differentiation/maturation, cell-cell interaction, cell-matrix interaction, cellular crosstalk/signaling, vascularization, etc.;
iv) stem cell research with focus on cellular differentiation and maturation as dispersed cells, spheroids, organoids, etc.

According to a second aspect, the present disclosure provides for a method of 3D bioprinting of a tissue model construct according to the above, comprising the steps of:

i) mixing cells with the bioinks, according to Cell A with Bioink A, and Cell B with Bioink B, in order to obtain a cell ladened Bioink A and a cell ladened Bioink B;

ii) transferring each of the obtained cell ladened Bioinks A and B to bioprinting cartridges A and B, respectively;

iii) mounting the bioprinting cartridges on printheads of a 3D bioprinter device;

iv) performing extrusion based bioprinting of the cell ladened bioinks, thereby obtaining at least one tissue construct, comprising at least two structures, wherein the first structure comprises Bioink A mixed with at least one Cell A, and the second structure comprises Bioink B mixed with at least one Cell B;

v) crosslinking of the bioprinted tissue to polymerize and/or gelate the tissue construct for culturing;

vi) culturing the tissue construct and optionally adding Factor A for stimulating or inducing phenotypical changes.

According to a third aspect, the present disclosure provides for a method of providing a tissue for implantation into a subject, said method comprising 3D bioprinting a tissue model construct according to the above. The method further comprises at least 48 hours of culturing the 3D bioprinted tissue in order to obtain a tissue that may be implanted into the subject.

According to a fourth aspect, the present disclosure provides for a method of treatment of a subject, said method comprising implanting a tissue obtained according to the third aspect above, into the subject.

According to a fifth aspect, the present disclosure provides for a kit for use in 3D bioprinting for generating a tissue model construct according to the above, said kit comprising:

i) a Bioink A comprising methacrylated gelatin or collagen with a liver specific ECM component, such as laminins, fibronectin or whole organ digest ECM from animal or human origin;

ii) a Bioink B comprising liver specific components. such as laminins, fibronectin or whole organ digest ECM from animal or human origin;

and optionally other components, such as Cell A and/or Cell B, and/or suitable antibodies, for use together with Bioink A and Bioink B.

Suitable antibodies may e.g. be antibodies to detect the phenotype, e.g. collagen type I antibody to detect deposition of collagen as a marker of abnormal function of stellate cells, CYP3A43, a cytochrome P450 family, which activity increases during drug metabolism by hepatocyte.

Thus, the present invention provides for combining a printable liver ECM bioink with the 3D bioprinting technology to create a functional liver tissue model for use in for example disease modelling. The combination of bioinks is designed to be used in connection with extrusion based bioprinting within the guidelines of provided protocols to generate a 3D liver tissue model. However, there is flexibility in parameters such as cell concentration, co-culture ratios, construct architecture, culture conditions, and concentration of induction factors that allows the user to tailor the liver model to be compatible with his or her investigation.

### BRIEF DESCRIPTION OF THE DRAWINGS

FIG. 1. Live primary hepatocytes in Bioink A in which some form clusters within the bioink.

FIG. 2. (A) LX2 in cellulose based bioink functionalized with laminin demonstrate cell-cell interaction as clusters. Primary stellate cells (B) as seen with Live/Dead staining and (C) multiphoton microscopy in GeIXA based bioink functionalized with animal-derived extracellular matrix exhibit cluster and stretched cell interaction and morphology, respectively.

FIG. 3. Live/dead images of HepG2 and LX2 co-cultures at (A) 2:1 and (B) 4:1 cell ratio in GeIXA based bioink functionalized with laminin.

FIG. 4. Actin and nuclear staining of HepG2 and LX2 co-cultures at (A) 2:1 and (B) 4:1 cell ratio in GeIXA based bioink functionalized with laminin.

FIG. 5. Total protein content of 3D bioprinted constructs cultured for 21 days with liver fibrosis induction. Co-culture (HepG2:LX2, 2:1 and 4:1 cell ratio) constructs, compared to HepG2 only constructs, exhibit higher of production of protein at all timepoints measured (day 12, 16, and 20) for higher relevance to native liver tissue.

### DEFINITIONS

“Bioprinting” refers to the utilization of 3D printing and 3D printing-like techniques to combine cells, growth factors, and biomaterials to fabricate biomedical parts that maximally imitate natural tissue characteristics. Generally, 3D bioprinting utilizes the layer-by-layer method to deposit materials known as bioinks to create tissue-like structures that are later used in medical and tissue engineering fields.

In this context, a “structure” may include a layer or any other 3D-printable three-dimensional construct, form or shape, that is amenable as a tissue model and especially a liver tissue model.

By “physiological conditions” are meant that that the culture or the cells are exposed to conditions (such as pH, osmolarity, temperature and printing pressure (which is equal to extrusion pressure in this context)) that are typical to the normal environment for the culture or cells, such as, for human cells, a temperature around 37° C., such as in the interval from 35-39° C., a printing pressure in the interval from 1 kPa to 200 kPa, preferably below 10 kPa, a pH in the interval from 5-8, preferably about 7, and an osmolarity in the interval from 275 to 300 mOsm/kg, preferably about 295 mOsm/kg.

By “pathological conditions” are meant that the culture or the cells are exposed to inflammatory and/or carcinogenic conditions, or other specific conditions to recapitulate a disease.

Spheroid is a set of cells confined within a spherical space having a predetermined diameter range, e.g. between 30 μm and 300 μm, preferably below 100 μm. The set of cells may be lumped together and/or embedded within an essentially spherical extracellular environment such as a gel or a bioink, whereon the spherical extracellular environment has a diameter within the predetermined range.

Methacrylated gelatin (GeIMA) is produced through the reaction of gelatin with methacrylic anhydride (MA). A large number of amino groups presenting on the side chains of gelatin are replaced by methacryloyl groups in methacrylic anhydride, forming modified gelatin. GeIMA obtains the feature of photocrosslinking because of the presence of methacryloyl groups. GeIMA hydrogel is capable of supporting cell behaviors and the biocompatibility and degradation property of gelatin have not been influenced. Furthermore, physical and chemical properties of GeIMA hydrogels can be tuned flexibly to meet the requirements of various applications.

### DETAILED DESCRIPTION

The tissue model construct disclosed herein comprises (i) at least two different bioinks that are optimized for hepatic cells of animal or human origin, (ii) hepatic cells which can be parenchymal and/or nonparenchymal, and (iii) optionally factors or compounds that can be employed to stimulate or induce phenotypical changes. The bioinks are based on naturally and/or synthetically derived biomaterials mixed with other components such as laminins, decellularized extracellular matrix, peptides, and other proteins that enhances cell attachment, proliferation, and maturation. The added components will be specific to the liver tissue in order to give the niche environment the various hepatic cells demand. In addition to the compatibility to encapsulate cells, the bioinks are optimized to have great printability, viscosity, and crosslinking capability. These bioinks will allow the user to generate complex 3D structures within the tissue model many types of 3D bioprinting technologies in which the user can innovate.

In one aspect, a kit is provided, acting as an instructional guide for a user to customize their specific experimental investigation using a 3D bioprinter. With the provided components, the user can determine which bioinks to use for which cells, the concentration of the cells within the bioink, the architecture of the construct, the localization of the cells, such as monoculture, co-culture, stimulating environment of migration, spatial organization of the cells and/or bioinks within the construct, and, last but not least, the culture conditions to enhance mimicry of physiological and/or pathological conditions. These parameters are optimized to ensure functionality of the liver model to be used as an assay platform or a developmental model. Accordingly is it possible to generate and obtain a model with normal or pathological tissue according to the specific experimental investigation to be performed.

The tissue model construct according to the present invention thus comprises i) a Bioink A; ii) a Bioink B; iii) a Cell A; and iv) a Cell B; and v) optionally a Factor A.

Bioink A and Bioink B may, each and independently of each other, comprise materials including but not limited to biopolymers synthetically or naturally derived from plants and/or animals, such as gelatin, alginate, cellulose, and thickening agents, photo initiators, and/or other extracellular matrix proteins. Thus, the bioinks are tailored to the tissue type in question in order to encourage the tissue maturation towards normal or abnormal liver function. The bioink is based on either a synthetic and/or natural biopolymer incorporated with extracellular matrix proteins that simulates the liver niche environment. The biopolymer can be a polysaccharide derived from botanical sources such as acacia gum, tara gum, glucomannan, pectin, locust bean gum, guar gum, carrageenan, and tragacanth. The bioink can have microbial or fungal-produced polysaccharide thickening agents known as biogums (such as xanthan gum, gellan gum, diutan gum, welan gum, and pullulan gum). Incorporation of extracellular matrix proteins derived from human and/or animal sources such as decellularized extracellular matrices, isolated laminins, and/or purified molecular proteins. Each component of the bioink is essential for printability, crosslinking, cellular attachment, cellular proliferation, cellular maturation, and cellular functionality. With the balanced niche provided by the bioinks, the hepatic cells of parenchymal and non-parenchymal lineage will maintain their respective physiological and pathological states as directed by the stimulating factors. The bioinks will support the cells through the bioprinting procedure in reducing shear stress experienced by the cells, in addition to providing spatial distribution for efficient nutrient and/or waste exchange.

The Bioink A may preferably comprise methacrylated gelatin or collagen with a liver specific Extra-Cellular Matrix (ECM) component. The ECM component may be for instance laminins, fibronectin or whole organ digest ECM from animal or human origin.

The Bioink B may preferably comprise liver specific components such as laminins, fibronectin or whole organ digest ECM from animal or human origin.

Combining a printable liver ECM bioink with the 3D bioprinting technology enables to create a functional liver tissue model for use in for example disease modelling.

Cell A and/or Cell B may, each and independently of each other, be immature or mature non-parenchymal hepatic cells originating from induced pluripotent stem cells, embryonic stem cells, any other stem cells, primary cells, and cell lines of human or animal origin.

Cell A may be a hepatic cell line of human or animal origin, or primary hepatic cells, or induced Pluripotent Stem cell(iPS)-derived or Embryonic Stem cell(ES)-derived hepatic cells. These cells, also referred to as hepatocytes, actively metabolize, respond, and transform the compound/substance of being tested. These cells are responsible for carbohydrate metabolism, lipid metabolism, and detoxication.

Cell B may be a non-parenchymal cell of human or animal origin from cell lines, primary cells, or derived from induced Pluripotent Stem cell s (iPSCs) or Embryonic Stem cells (ESCs). Non-parenchymal cells include liver sinusoidal endothelial cells, Kupffer cells, biliary cells, lymphocytes, and hepatic stellate cells in normal and abnormal physiology. These cells, also referred to as helper cells, are responsible for filtration of macromolecules, may act as macrophages, or may act as myofibroblasts.

The cells included in the tissue model are derived from preferably a human source and/or animal sources isolated from liver tissues or derived from stem cells, such as embryonic or induced pluripotent stem cells, which are models that are for the functionality of hepatocytes and for testing of compound toxicity and metabolic effects on such cells or tissue models comprising such cells. Cell lines such as HepG2 and HepaRG are hepatocytes commonly used to investigate albumin production, CYP activity, and toxicity of drug compounds on 2D in vitro cultures. Primary cells are also commonly used, but at a lower throughput because of the complicated sandwich cultures, and the most prominent feature of using such primary cells is the short working time of 2-96 hours after cell seeding. For more developed models, stellate cells are incorporated to investigate fibrotic conditions of the liver in 2D. For more 3D spheroid advanced models, a heterogenous mixture of primary non-parenchymal cells, such as Kupffer cells, sinusoidal endothelial cells, biliary epithelial cells, and stellate cells, are combined with primary hepatocytes. The spheroid models have a longer working window once the spheroid has been formed, which can take up to 3 days. By using 3D bioprinting, providing the tissue model construct of the present invention, the 3D model generated will utilize less cells than spheroid culture and the cells are not compact and confined to the spheroid architecture. Different variations with either cell lines or primary cells may be provided for the user to conduct their experiment in 3D in vitro conditions. With 3D bioprinting, arrangement of the different cell types in relation to each other can be defined by the user for the specific question in mind. For example, hepatocytes as Cell A can be bioprinted in Bioink A as a first layer upon which non-parenchymal cells as Cell B, encapsulated in a Bioink B, are bioprinted to investigate the cell-cell migration behavior, the paracrine communication, and the functionality of the cells within the tissue model. Cell A and Cell B can be used at different ratios in relation to each other, in order to simulate a pathology in vitro.

To stimulate pathological metabolism of the cells as mentioned above, various factors, corresponding to Factor A in the present disclosure, have been used in current investigations such as TGF-β, free fatty acids, lipopolysaccharide in combination with allyl alcohol, cytokines such as interleukins and tumor necrosis factors, and other chemical/molecular agents. Each factor has been shown to affect and target a specific cell type. For example, TGF-β activates the stellate cells to increase production of collagens, which in turn induces fibrosis. Free fatty acids activate lipo-apotosis of hepatocytes, leading to non-alcoholic fatty liver disease. Allyl alcohols in combination with lipopolysaccharide cause hepatoxicity by interfering with Kupffer cell mechanisms. Tumor necrosis factor-alpha is a pro-inflammatory factor with has an effect in various liver pathologies with limited mechanistic understanding. The concentration and/or combinations of the factor(s) can vary to stimulate the level of severity of a disease or pathological condition, the specific stage of a disease, and the metabolic pathway of choice intended to be investigated. Hence, the tissue model that the user can generate by use of the present invention can simulate fatty liver disease, liver fibrosis, non-alcoholic liver disease, liver cancer, cirrhosis, and other liver metabolic syndromes. The tissue models can then be used to study the development of the disease, the etiology, and the treatment and/or reduction of an abnormal physiology.

The combination of functionalized bioinks according to the present disclosure is a novel aspect, since the bioinks are tailored to hepatic cells to promote liver tissue mimicry. The bioinks are formulated from synthetically and naturally derived biopolymers, macromolecules, proteins, and small molecules from plants, microbial, animals, and/or human sources. Biopolymers include but are not limited to polysaccharides, extracellular matrix proteins derived from animal/human tissues, such as glycosaminoglycans, collagens, elastins, proteoglycans, laminins, aggrecans. Laminins, fibronectin or whole organ digest ECM from animal or human origin may be preferred. The polysaccharides are preferably comprised of cellulose, and may have different fibrillar structures. Cellulose can be generated from plants (such as annual plants), trees, fungi or bacteria, and are preferably generated from bacteria such as from one or more of the genera Aerobacter, Acetobacter, Acromobacter, Agrobacterium, Alacaligenes, Azotobacter, Pseudomonas, Rhizobium, and/or Sarcina, specifically Gluconacetobacter xylinus, Acetobacter xylinum, Lactobacillus mali, Agrobacterium tumefaciens, Rhizobium leguminosarum bv. trifolii, Sarcina ventriculi, enterobacteriaceae Salmonella spp., Escherichia coli, Klebsiella pneu-moniae and several species of cyanobacteria. Cellulose can be generated from any vascular plant species, which include those within the groups Tracheophyta and Tracheobionta.

According to one embodiment, the cellulose used is cellulose nanofibrils. The advantage of using cellulose nanofibril hydrogels is extreme shear thinning properties which is crucial for high printing fidelity. Cellulose nanofibrils formed from cellulose producing bacteria most closely mimic the characteristics of collagen found in human and animal soft tissue. The array of fibrils provides a porous yet durable and flexible material. The nanofibrils allow nutrients, oxygen, proteins, growth factors and proteoglycans to pass through the space between the fibrils, allowing the scaffold to integrate with the implant and surrounding tissue. The nanofibrils also provide the elasticity and strength needed to replace natural collagen. The bacterial cellulose materials have been, after purification, homogenized and hydrolyzed to smooth dispersion. WO2016/100856 is hereby incorporated as a reference for the cellulose-based bioink material.

Wood-derived cellulose nanofibrils can be selected as an alternative raw material for the preparation of cellulose nanofibrillated bioink. The difference is that they do not form three dimensional networks and their width is lower (10-20 nanometers) and length is lower (1-20 micrometers). The disadvantage of the wood derived cellulose nanofibrils can be the presence of other wood biopolymers such as hemicelluloses which can affect cells and cause foreign body reaction. These dispersions should preferably therefore be purified by an extraction process and removal of the water phase. It is a sensitive process since it can lead to agglomeration of fibrils which can result in bioink which tends to clog the 3D bioprinter printing nozzle. Preferably such a dispersion should also be homogenized and subject to ultrafiltration to prepare a bioink.

The bioinks formulations according to the present invention may comprise other components to enhance printability, viscosity, crosslinking capability, degradation, and cellular metabolism/activity. For instance at least one additional biopolymer may be added to the bioink, wherein the biopolymer gelling agent or hydrocolloid is chosen from the group comprising alginates, hyaluronic acid and its derivatives, agarose and its derivatives, chitosan, fibrin, gellan gum, crystalline nanocellulose, carrageenans, collagen and its derivatives as well as gelatin and its derivatives. These additional biopolymers are added to the bioink for crosslinking purposes and/or to contribute to rheological properties as hydrocolloids or thickening agents. Addition of crosslinker or binding biopolymers such as alginate can be used to improve printability but also provide mechanical stability after crosslinking.

As specified above, each component of the bioink is essential for i.a. printability. Preferably at least one of the bioinks A and B comprises a methacrylated gelatin. By using a methacrylated gelatin, the mechanical stability of a construct produced by bioprinting with the Bioinks A and/or Bioinks B is enhanced. Furthermore, by comprising methacrylated gelatin in at least one of the Bioinks A and B, it is possible to cross-link the constructs, which will further enhance the mechanical stability of the construct.

Additionally, by incorporating thickening agents in the Bioink A and in the Bioink B, the rheological properties of the bioinks is improved by increasing the viscosity of the bioinks. This will in turn improve the bioprinting process as the bioprinting shape fidelity is improved. In this aspect, bioprinting shape fidelity means that the bioprinted construct will keep the shape as is printed, and will not lose said shape after having been bioprinted.

Furthermore, the thickeners (thickening agent) improve the shear thinning properties of the bioinks. An increased viscosity provided by the thickeners will protect the cells from shear stress during the bioprinting process. The thickeners will provide a window shift for the gelation point leading to less temperature dependence during the bioprinting process. More specifically, the window shift moves the printability temperature to the 20-24° C. range, which is much easier to achieve. The bioprinting can be performed without temperature-controlled printheads.

The thickening agents may be natural or synthetic. Preferably, the thickening agent is a natural polysaccharide, which is preferably chosen from a group comprising xanthan gum, glucomannan and nanocellulose. Xanthan gum, glucomannan and nanocellulose have been shown to be particularly advantageous thickening agents in bioinks for 3D bioprinting applications. Advantages provided are for example that such agents modify the viscosity, shift the gelation temperature, and improve the printing resolution of complex multi layer structures.

The bioinks provided will have unique capacities to support the metabolism and functionality of the cell types of interest. By functionalizing the bioink with liver specific laminins, liver specific extracellular matrix proteins derived from livers, and other macromolecules, such as exosomes, proteins, ligands, factors isolated/extracted from different animal/human tissues, a niche environment will be created which support cell lines, stem cells (ESCs or iPSCs), and primary cells of both animal and human origin. The livers from which liver specific extracellular matrix proteins are derived may be of animal or human origin, and may be derived from livers of different conditions, such as age and any disease, and may also be derived using different extraction methods.

The employed cells will preferably be of human origin in order to elevate the relevance of the 3D liver tissue model especially for pre-clinical based applications in order to facilitate the translation to clinical trials and/or simulate human response in order to limit animal testing. These cells can be of human or animal origin, it may relate to cell lines, primary cells, and heterogenous mixtures of cells, which are currently utilized within the field of liver research. Cell lines include but are not limited to HepG2 (carcinoma hepatocytes) and HepaRG (stem cell derived), cell lines which are commonly utilized as a model for liver metabolic function. For elevated human relevance, primary hepatocytes, isolated from livers of different donors with variations in age, gender, and condition, can also be used. However, these primary hepatocytes are more challenging to maintain. Liver stellate cells such as LX-1 and LX-2 are commonly used for their preserved and controlled activated/non-activated stellate characteristics. Primary stellate cells, isolated from donors of different age, gender, and condition, will provide a mimicry complexity to the tissue model without the handling challenges. A heterogenous mixture of non-parenchymal cells are commonly incorporated to elevate the metabolic functionality of hepatocytes to simulate in vivo like cellular mechanisms.

To direct the liver tissue models towards pathological metabolism, chemical stimulants are necessary. Hence, one or more factors to allow the user to simulate different phases and conditions of liver abnormality may be used on the tissue model construct. Common factors used in the field are TGF-β1, free fatty acids, and other molecules can drive for example, over production of extracellular matrix by the stellate cells and the accumulation of lipid vesicles by the hepatocytes, respectively. Thus, a Factor A, as disclosed above, may be included according to the invention, in order to stimulate the bioprinted tissue to develop into the kind of hepatic tissue, normal or abnormal, that is required for the experimental investigation to be performed.

According to one embodiment, Bioink A and Bioink B, each and independently of each other, comprises:
a plant based material 0-90%
an animal/human derived material 0-90%
thickening agents 0-50%
a photo-initiator 0-10%
matrix proteins and/or protein components 0-20%.

In another embodiment, Bioink A and Bioink B, each and independently of each other, comprises:
a plant based material 0-50%
an animal/human derived material 0-50%
thickening agents 0-50%
a photo-initiator 0-10%
matrix proteins and/or protein components 0-20%.

Bioink A and Bioink B may have the same proportions of the components mentioned above, or they may have different proportions of the components mentioned above.

In one embodiment, a plant based material and/or an animal/human derived material, such as ECM material (ECM: extracellular matrix), may be added with thickener only.

Examples of the plant based materials and the animal/human derived materials are as disclosed above.

The thickening agents may be synthetic or natural as disclosed above.

The photo-initiator is a molecule that generates reactive species when exposed to radiation, UV or visible, and facilitates polymerization of the bioink. The photo-initiator may be for instance lithium phenyl-2,4,6-trimethylbenzoylphosphinate and/or irgacure.

The matrix proteins and/or protein components may be collagen, elastin, proteoglycans, glycoaminoacids, and laminins. The matrix proteins and/or protein components can be well defined in the bioink to support specifically parenchymal and/or non-parenchymal cells. For example, one may have only one or two components of the matrix protein, which are specific for the particular cell type. E.g. one can use laminin 111 for hepatocytes.

The tissue model construct of the present invention may thus be for use within science, medicine, tissue engineering, pharmaceutical therapies, regenerative medicine, stem cell research, and in vitro models. It may further relate to use for 3D bioprinting and/or construction of desired tissue models.

Additionally there is provided a method of bioprinting the tissue model construct according to the above, wherein the 3D bioprinting is performed with an extrusion base bioprinting device.

The tissue model construct according to the present invention may be used for
i) Developmental biology in order to gain understanding of cellular activities within a 3D environment such as cellular distribution, migration, proliferation, matrix production, interactions with other cells and the surrounding environment, etc.;
ii) Pharmaceutical applications for drug discovery, target validation, toxicity studies, metabolism studies, cellular differentiation/maturation, spheroid differentiation/maturation, organoid differentiation/maturation, etc.;
iii) Tissue regeneration applications such as tissue remodeling, cellular proliferation, cellular metabolism, cellular differentiation/maturation, cell-cell interaction, cell-matrix interaction, cellular crosstalk/signaling, vascularization, etc.;
iv) Stem cell research with focus on cellular differentiation and maturation as dispersed cells, spheroids, organoids, etc.

The present disclosure also provides for a method of 3D bioprinting of a tissue model construct according to the above comprising the general steps of

(a) mixing the cells with the bioinks,

(b) choosing the desired design parameters (architecture, culture plates) of the liver tissue,

(c) bioprinting models with reproducibility,

(d) crosslinking the bioprinted constructs,

(e) culturing the liver tissue constructs with and/or without induction factor

Thereafter samples are collected from the liver tissue culture for analysis.

For example, the 3D bioprinting is performed with an extrusion based bioprinting device.

The method of 3D bioprinting of a tissue according to the above more specifically may comprise the steps of:

i) mixing cells with the bioinks, according to Cell A with Bioink A, and Cell B with Bioink B, in order to obtain a cell ladened Bioink A and a cell ladened Bioink B;

ii) transferring each of the obtained cell ladened Bioinks A and B to bioprinting cartridges A and B, respectively;

iii) mounting the bioprinting cartridges on printheads of a 3D bioprinter device;

iv) performing extrusion based bioprinting of the cell ladened bioinks, thereby obtaining tissue constructs;

v) crosslinking of the bioprinted tissue to polymerize and/or gelate the tissue construct for culturing;

vi) culturing the tissue construct and optionally adding Factor A for stimulating or inducing phenotypical changes.

Preferably the bioprinted tissue model construct is an hepatic tissue.

The protocols provided serve as examples for designing liver models. For example, a kit and protocol for liver fibrosis may be provided utilizing Bioink A (cellulose based bioink comprising laminin) with Cell A (primary hepatocytes), Bioink B (cellulose based bioink comprising laminin 521) with Cell B (primary stellate cells), and Factor A (TGF-β). The exemplary protocol comprises the following steps:
i) Bioinks, at least 1 ml of Bioink A and 1 ml of Bioink B, are warmed to 37° C.
ii) Cells, that are Cells A and Cells B, are harvested following manufacturer's protocols and condensed to 100 μl with 10 million cells for primary hepatocytes and 100 μl with 10 million cells for primary stellate cells, in their respective medium as recommended for the specific cell type;
iii) Bioink A, 1 ml, is mixed with Cell A, 100 μl;
iv) Cell ladened Bioink A obtained in iii) is transferred to a cartridge A before placing the cartridge A into a printhead of a 3D bioprinter;
v) Bioink B, 1 ml, is mixed with Cell B, 100 μl;
vi) Cell ladened Bioink B obtained in v) is transferred to a cartridge B before placing the cartridge B into a second printhead of the 3D bioprinter;
vii) A bioprinting program is chosen for the desired construct architecture with a first layer of Bioink A and a second layer of Bioink B;
viii) The bioprinter is calibrated to bioprint tissue constructs into well plates, such as 96-, 48-, or 24-well plates;
ix) Once the bioprinting is complete, crosslinking can be performed using a crosslinking solution or UV;
x) Culture medium is dispensed on the bioprinted tissue constructs for cell culturing.

Preferably, the mixing of a bioink with a cell is performed using sterile syringes connected with a luer connector.

Preferably a cell ladened bioink is transferred to a cartridge by using a luer connector, and thereafter closing said cartridge with a nozzle.

With 1 ml of cell ladened bioink it is possible to bioprint 50 constructs using 20 μl of each cell ladened bioink.

The induction of fibrosis in the cell culture obtained above with TGF-β can be performed at desired timepoints by adding the desired concentration to the culture medium. The skilled person will be familiar of the concentrations to be added to the culture medium in order to achieve an effect on said cultured cells.

Samples may thereafter be collected from the cell culture at specified and/or predetermined timepoints for analysis.

Furthermore, the present disclosure provides for a method of providing tissue for treatment of a subject, said method comprising 3D bioprinting of a tissue model construct as disclosed herein. For this purpose, the method further requires at least two days (48 hours) of culture after bioprinting and crosslinking, to ensure cell recovery within the bioprinted tissue before said tissue may be implanted into a subject in need of treatment.

Furthermore, the present disclosure provides for treatment of a subject comprising implanting a tissue, produced by the methods as disclosed above and consequently implanting said tissue into the subject. Preferably the method of treatment relates to implanting of a liver tissue into a subject.

The present disclosure also provides for a kit for use in 3D bioprinting for generating a tissue model construct according to the above, said kit comprising:
i) a Bioink A comprising methacrylated gelatin or collagen with a liver specific ECM component, such as laminins, fibronectin or whole organ digest ECM from animal or human origin;
ii) a Bioink B comprising liver specific components. such as laminins, fibronectin or whole organ digest ECM from animal or human origin;

and optionally other components, such as a Factor A, and/or Cell A and/or Cell B, for use together with Bioink A and Bioink B.

In one embodiment, the kit further comprises a thickening agent, either separately or included in Bioink A and/or B.

The compositions of the Bioinks A and B in the kit are as specified above. Preferably the Bioink A may comprise methacrylated gelatin or collagen with a liver specific Extra-Cellular Matrix (ECM) component, such as laminins, fibronectin or whole organ digest ECM from animal or human origin. Preferably the Bioink B may comprise liver specific components such as laminins, fibronectin or whole organ digest ECM from animal or human origin. Preferably the Bioink A and/or the Bioink B comprises a thickening agent that is a natural polysaccharide. Preferably the thickening agent is chosen from a group comprising xanthan gum, glucomannan and nanocellulose. Providing such a kit enables combining a printable liver ECM bioink with the 3D bioprinting technology to create a functional liver tissue model for use as disclosed above.

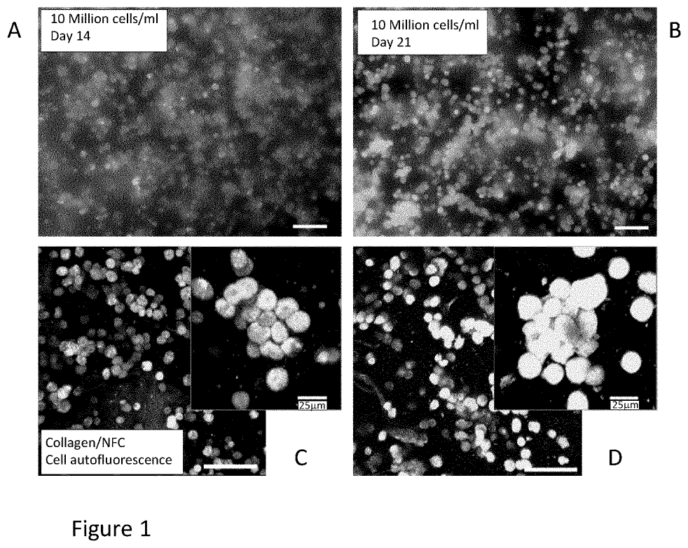

FIG. 1 discloses live primary hepatocytes as Cell A in a Bioink A at A) day 14, and b) day 21. When studying the cell cultures using cell autofluorescence (FIGS. 1C and 1D), it can be seen that some of the hepatocytes have formed loose aggregates for cell-cell communication similar to the native cell-cell contact within the bioink at both day 14 and day 21.

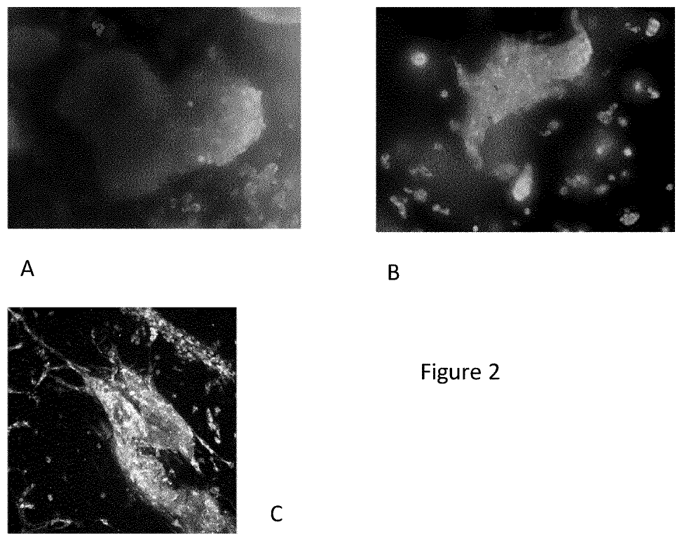

FIG. 2A discloses human hepatic stellate cell line LX2 in cellulose based bioink functionalized with laminin, demonstrating cell-cell interaction as clusters. FIGS. 2B and 2C discloses primary stellate cells in GeIXA based bioink functionalized with animal-derived extracellular matrix, exhibiting cluster and stretched cell interaction and morphology, respectively. The cells thus exhibits a good cell viability.

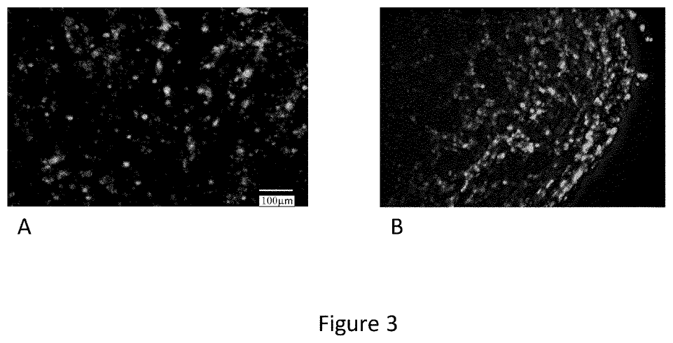

FIG. 3 discloses images of live and dead cells of HepG2 and LX2 under co-cultures at (A) 2:1 and (B) 4:1 cell ratio in GeIXA based bioink functionalized with laminin. FIG. 3 thus illustrates the cell viability for the cells incorporated in the co-cultures

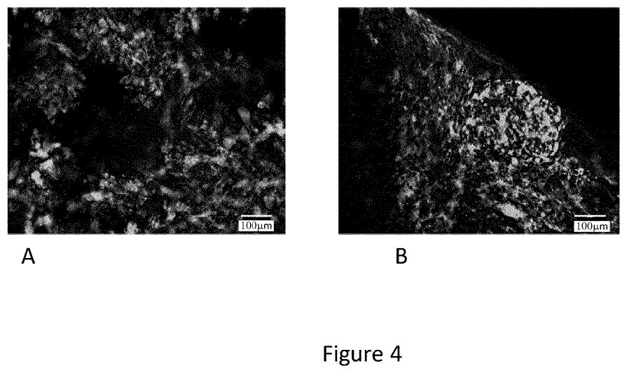

FIG. 4 discloses actin and nuclear staining of HepG2 and LX2 co-cultures at (A) 2:1 and (B) 4:1 cell ratio in GeIXA based bioink functionalized with laminin. FIG. 4 thus illustrates the organisation of the viable cells within the co-cultures.

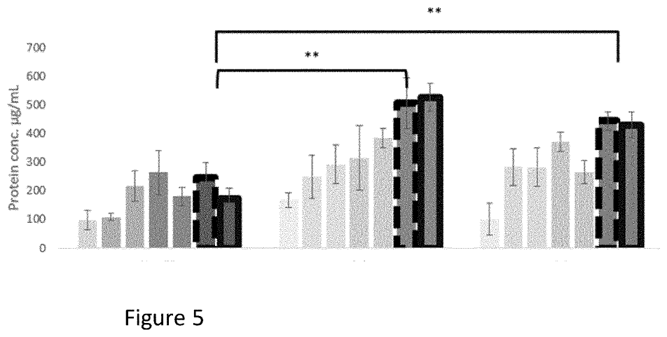

FIG. 5 shows diagrams of the total protein content of 3D bioprinted constructs cultured for 21 days with liver fibrosis induction. Co-culture (HepG2:LX2) constructs at cell ratios 2: and 4:1 exhibit higher production of protein for higher relevance to native liver tissue compared to HepG2. For each set, three bars are shown corresponding to 12, 16 and 20 days, from the left to the right. Thus, tissues of co-culturing of hepatic cells and stellate cells leads to a higher protein content than tissues comprising only hepatic cells after induction of liver fibrosis.

1. A liver tissue model construct comprising at least one bioprinted structure, wherein the structure comprises a Bioink A mixed with at least one Cell A, and a Bioink B mixed with at least one Cell B,
wherein at least one of Bioink A and Bioink B, independently of each other, is based on methacrylated gelatin, collagen, nanocellulose and/or alginate;
wherein at least one of Bioink A and Bioink B comprises liver-specific ECM components;
optionally wherein at least one of Bioink A or Bioink B comprises a thickening agent, wherein the thickening agent is a natural polysaccharide selected from the group consisting of: xanthan gum, glucomannan and nanocellulose;
wherein at least one of Bioink A or Bioink B comprises a Factor A;
wherein Cell A is a hepatic cell line of human or animal origin; and
wherein Cell B is a non-parenchymal cell of human or animal origin from cell lines, primary cells, or derived from induced pluripotent stem cells (iPSCs) or embryonic stem cells (ESCs), wherein the non-parenchymal cells are selected from the group consisting of: liver sinusoidal endothelial cells, Kupffer cells, biliary cells, lymphocytes, and hepatic stellate cells in normal and abnormal physiology.

2. The liver tissue model construct according to claim 1, wherein the Bioink A comprises methacrylated gelatin or collagen with a liver specific ECM component.

3. The liver tissue model construct according to claim 1, wherein Bioink B comprises liver specific components.

4.-6. (canceled)

7. The liver tissue model construct according to claim 1, wherein the Factor A is selected from the group consisting of: TGF-β, free fatty acids, cytokines, interleukins, tumor necrosis factors, and lipopolysaccharide in combination with allyl alcohol.

8. The liver tissue model construct according to claim 1, wherein Bioink A and Bioink B, each and independently of each other, comprises:
methacrylated gelatin 0-90%;
alginate 0-90%;
liver specific ECM components 0-50%;
thickening agents 0-50%;
a photo initiator 0-10%; and
a growth factor specific to parenchymal cells 0-20%.

9. The liver tissue model construct according to claim 1, wherein Cell A and Cell B are used at different ratios in relation to each other, in order to simulate a pathology in vitro.

10. The liver tissue model construct according to claim 1, wherein the liver tissue model construct is suitable for use within science, medicine, tissue engineering, pharmaceutical therapies, regenerative medicine, stem cell research, and in vitro models.

11. Method of bioprinting the liver tissue model construct of claim 1, comprising 3D bioprinting the liver tissue model construct with an extrusion based bioprinting device.

12. The liver tissue model construct according to claim 1, wherein the liver tissue model construct is suitable for use in:
i) Developmental biology in order to gain understanding of cellular activities within a 3D environment such as cellular distribution, migration, proliferation, matrix production, interactions with other cells and the surrounding environment;
ii) Pharmaceutical applications for drug discovery, target validation, toxicity studies, metabolic studies, cellular differentiation/maturation, spheroid differentiation/maturation, and organoid differentiation/maturation;
iii) Tissue regeneration applications such as tissue remodeling, cellular proliferation, cellular metabolism, cellular differentiation/maturation, cell-cell interaction, cell-matrix interaction, cellular crosstalk/signaling, and vascularization; and/or
iv) Stem cell research with focus on cellular differentiation and maturation as dispersed cells, spheroids, and organoids.

13. Method of 3D bioprinting of a liver tissue model construct of claim 1, comprising:
i) mixing cells with the bioinks, according to Cell A with Bioink A, and Cell B with Bioink B, in order to obtain a cell ladened Bioink A and a cell ladened Bioink B,
ii) transferring each of the obtained cell ladened Bioinks A and B to bioprinting cartridges A and B, respectively;
iii) mounting the bioprinting cartridges on printheads of a 3D bioprinter device;
iv) performing extrusion based bioprinting of the cell ladened bioinks, thereby obtaining at least one tissue construct comprising at least one structure, wherein the structure comprises Bioink A mixed with at least one Cell A, and Bioink B mixed with at least one Cell B;
v) crosslinking of the bioprinted tissue to polymerize and/or gelate the tissue construct for culturing; and
vi) culturing the tissue construct and optionally adding Factor A for stimulating or inducing abnormal changes.

14. Method of providing a tissue for implantation into a subject, said method comprising 3D bioprinting of a liver tissue model construct of claim 1, wherein the method further comprises at least 48 hours of culturing the 3D bioprinted tissue in order to obtain a tissue that may be implanted into the subject.

15. Method of treatment of a subject, said method comprising implanting a liver tissue obtained according to the method of claim 14 into the subject.

16. A kit for use in 3D bioprinting for generating a liver tissue model construct according to claim 1, said kit comprising:
i) a Bioink A, and
ii) a Bioink B,
wherein at least one of Bioink A and Bioink B comprises methacrylated gelatin, collagen, alginate or nanocellulose;
wherein at least one of Bioink A and Bioink B comprises liver-specific ECM components;
optionally wherein at least one of Bioink A or Bioink B comprises a thickening agent, wherein the thickening agent is a natural polysaccharide selected from the group consisting of: xanthan gum, glucomannan and nanocellulose;
wherein at least one of Bioink A or Bioink B comprises a Factor A;
and optionally other components selected from the group consisting of: Cell A and/or Cell B, and/or suitable antibodies, for use together with Bioink A and Bioink B.

17. (canceled)

18. The liver tissue model construct according to claim 1, wherein (1) at least one of Bioink A and Bioink B is based on methacrylated gelatin with no addition of thickening agent, or (2) at least one of Bioink A and Bioink B is based on alginate with addition of nanocellulose as thickening agent.

19. The liver tissue model construct according to claim 1, comprising at least two 3D bioprinted structures, wherein the first structure comprises the Bioink A mixed with at least one Cell A, and the second structure comprises the Bioink B mixed with at least one Cell B.

20. The method according to claim 13, wherein iv) comprises obtaining at least one tissue construct comprising at least two structures, wherein the first structure comprises the Bioink A mixed with at least one Cell A, and the second structure comprises the Bioink B mixed with at least one Cell B.

21. The liver tissue model construct of claim 1, wherein the liver-specific ECM components are selected from the group consisting of: laminins, fibronectin, and whole organ digest extracellular matrix (ECM) from animal or human origin.

22. The liver tissue model construct of claim 2, wherein the liver-specific ECM component is selected from the group consisting of: laminins, fibronectin, and whole organ digest ECM from animal or human origin.

23. The liver tissue model construct of claim 3, wherein the liver-specific components are selected from the group consisting of: laminins, fibronectin, and whole organ digest ECM from animal or human origin.

24. The liver tissue model construct of claim 9, wherein the different ratios are in an interval from 2:1 to 4:1 (Cell A:Cell B).

25. The kit of claim 16, wherein the liver-specific ECM components are selected from the group consisting of: laminins, fibronectin, and whole organ digest ECM from animal or human origin; and wherein the Factor A is selected from the group consisting of: TGF-β, free fatty acids, cytokines, interleukins, tumor necrosis factors, and lipopolysaccharide in combination with allyl alcohol.
